(19) United States
(12) Patent Application Publication
(10) Pub. No.: US 2025/0322523 A1
(43) Pub. Date: Oct. 16, 2025

(54) METHODS AND SYSTEMS FOR VERIFYING LOADS FOR BIODECONTAMINATION

(71) Applicant: Ecolab USA Inc., St. Paul, MN (US)

(72) Inventors: Christopher Moen Flynn, Saint Paul, MN (US); Grant Daniel Lindh, Woodbury, MN (US); Albert GoldFain, Chanhassen, MN (US); Oliver Cumberlege, Andover (GB); Kostas Tsekouras, Aurora, IL (US)

(21) Appl. No.: 19/175,801

(22) Filed: Apr. 10, 2025

Related U.S. Application Data
(60) Provisional application No. 63/632,331, filed on Apr. 10, 2024.

(51) Int. Cl. G06T 7/00; A61L 2/24; H04N 23/63

(52) U.S. Cl. CPC G06T 7/0014; A61L 2/24; H04N 23/632; A61L 2202/14

(57) ABSTRACT

The disclosure includes a system comprising a load comprising at least a first object in a first region and a second object in a second region. The system further includes one or more sensors and a computing device configured to control the one or more sensors to capture one or more images of the load including at least the first region and the second region. For a first area in the one or more images that includes the first region, the computing device applies a first set of rules to verify that the first object is properly arranged and, in response to determining that the first object violates at least one rule in the first set of rules, outputs an indication that the first object is not properly arranged. The computing device may repeat this process for the second area in the one or more images.

### RELATED APPLICATION

This application claims the benefit of U.S. Provisional Application No. 63/632,331, filed Apr. 10, 2024, the entire contents of which are incorporated herein by reference.

### TECHNICAL FIELD

The disclosure relates to biodecontamination procedure analysis.

### BACKGROUND OF THE INVENTION

For many applications within the life science industry, an aseptic transfer of products is critical to prevent the contamination of product leading to the infection of patients or operators. There are many examples where it is critical that items must be transferred into an aseptic environment and thus be biodecontaminated, or where items must be biodecontaminated on the way out of a process to prevent contamination of the operators or environment where the process is using live organisms.

Typical examples of the requirement to biodecontaminate items on the way into a process, is the transfer of items from a Grade C to a Grade B pharmaceutical clean room, where the product is to be used as part of the manufacturing or compounding process in a pharmaceutical facility or hospital pharmacy. Similarly, when transferring into a pharmaceutical isolator, Restricted Access Barrier System (RABS) or similar device where the preparation, filling or compounding of pharmaceutical products are taking place whether on a small, single item level or a continuous system filling/processing thousands of items an hour. Another typical application is the placing of items into an enclosure where contamination could adversely affect important results such as sterility testing, or during research with everything from simple organisms through to animals.

In addition, it can be necessary to ensure that items are biodecontaminated on the way out of a process which is using live organisms, such as vaccine manufacture or the use of viral vectors. In this case, the environment and operators outside the process must not become contaminated with the organism used in the process.

The biodecontamination can take place in a dedicated chamber which is entered and exited from a clean room or clean rooms, or as an entry or exit hatch to a pharmaceutical isolator, RABS, or similar type of enclosure. Alternatively, the whole isolator or RABS can be biodecontaminated with the items already placed inside.

There are a number of methods to biodecontaminate items, however, those requiring a gaseous, vapor, fogging, or UV light system, the placement of the items is critical. Before the routine use of the biodecontamination process within a chamber, validation must be done. Validation is a systematic series of studies which determines the placement of items within the chamber and the setting of the biodecontamination parameters to obtain a pre-determined level of kill, usually measured by the log reduction of a test organism placed on coupons within the chamber.

It is important that thereafter the items, known as a load, are placed in identical positions to be sure that the same level of kill is achieved as was proved during validation. Critically the amount of occlusion must be minimized, as may happen when two items touch each other, as these areas may be shielded from the decontaminant and thus viable microorganisms may remain.

As stated the location of the items in the load is critical so the current process is to visually compare the load configurations to printed images of the validated configuration, however this is a manual, subjective, and error prone process. In addition, there is a growing demand from regulators for a record of the chamber loading as a means to check and also for conducting of investigations if there is a contamination event. At present the only practical means is to use photographs or video. These images are often obstructed, blurry, or of insufficient resolution to analyze effectively. Due to the importance of placing the load into the chamber correctly, often a second person is required to check that it has been done correctly.

### SUMMARY OF THE INVENTION

In general, the disclosure describes methods and systems for automatically analyzing contents arranged for a chamber which will be biodecontaminated to determine whether the items have been located in their correct position. The position of items in their validated positions can be determined inside or outside of a biodecontamination chamber; for example, on a cart, tray, conveyer, or bin to be transferred into a biodecontamination chamber while maintaining the relative position of the objects. Using one or more images from one or more sensors, such as any combination of a camera, cameras, LiDAR sensors, or other vision system, a computer system will cycle through each of the various regions of a field of view where the load is placed prior to its biodecontamination, applying the particular rules applicable to that region, to determine whether the chamber is properly arranged. For the purposes of this disclosure, a “load” is considered to be any arrangement of objects that may be placed, or is already placed, in a device that is configured to perform a biodecontamination process, regardless of whether that arrangement of objects is valid, invalid, complete, or incomplete/partial. For the purposes of this disclosure, a load being “properly arranged” means that objects in that load, when compared to the one or more rules defining a validated load, are present in one or more images, that the objects are in the expected positions, both absolutely and relative to other objects that will ultimately be in the biodecontamination chamber, and that no objects that are not supposed to be in the biodecontamination chamber are present in the chamber (e.g., when comparing images of a load and a predetermined valid load, the images may be within a threshold of an image difference metric). If the system determines that the items are not properly arranged, the system may output some notification or warning to notify the user prior to proceeding with the biodecontamination process.

Rather than a manual comparison to a printed picture, the digital vision system described herein, using an integrated set of one or more sensors, such as one or more cameras, LiDAR sensors, or other vision systems, may provide a standardized load quality record. Additionally, the techniques described herein may quickly and accurately assess load compliance, increasing consistency and decreasing the amount of time users spend loading and checking the load for the biodecontamination process. Furthermore, using the techniques described herein may remove the need for a second person to check the load's position enabling them to focus on other activities and increasing the efficiency of the entire process.

In one example, the disclosure is directed to a method in which one or more processors control one or more sensors to capture one or more images of a load, wherein the load contains at least a first object in a first region of the load and a second object in a second region of the load, wherein the one or more images each include at least the first region of the load and the second region of the load. The method further includes, for a first area in the one or more images that includes the first region of the load, applying, by the one or more processors, a first set of rules to the first area in the one or more images to verify that the first object is properly arranged for a biodecontamination process, and, in response to determining that the first object violates at least one rule in the first set of rules, outputting, by the one or more processors and to an output device, an indication that the first object is not properly arranged for the biodecontamination process. The method also includes, for a second area in the one or images that includes the second region of the load, applying, by the one or more processors, a second set of rules to the second area in the one or more images to verify that the second object is properly arranged for the biodecontamination process, wherein the second set of rules is different than the first set of rules, and, in response to determining that the second object violates at least one rule in the second set of rules, outputting, by the one or more processors and to the output device, an indication that the second object is not properly arranged for the biodecontamination process.

In another example, the disclosure is directed to a system comprising one or more sensors. The system also includes a computing device comprising one or more processors configured to control one or more sensors to capture one or more images of a load, wherein the load contains at least a first object in a first region of the load and a second object in a second region of the load, wherein the one or more images each include at least the first region of the load and the second region of the load. The one or more processors are further configured to, for a first area in the one or more images that includes the first region of the load, apply a first set of rules to the first area in the one or more images to verify that the first object is properly arranged for a biodecontamination process, and, in response to determining that the first object violates at least one rule in the first set of rules, output, to an output device, an indication that the first object is not properly arranged for the biodecontamination process. The one or more processors are also configured to, for a second area in the one or images that includes the second region of the load, apply a second set of rules to the second area in the one or more images to verify that the second object is properly arranged for the biodecontamination process, wherein the second set of rules is different than the first set of rules, and, in response to determining that the second object violates at least one rule in the second set of rules, output, to the output device, an indication that the second object is not properly arranged for the biodecontamination process.

In another example, the disclosure is directed to a non-transitory computer-readable storage medium containing instructions. The instructions, when executed, cause one or more processors to control one or more sensors to capture one or more images of a load, wherein the load contains at least a first object in a first region of the load and a second object in a second region of the load, wherein the one or more images each include at least the first region of the load and the second region of the load. The instructions, when executed, further cause one or more processors to, for a first area in the one or more images that includes the first region of the load, apply a first set of rules to the first area in the one or more images to verify that the first object is properly arranged for a biodecontamination process, and, in response to determining that the first object violates at least one rule in the first set of rules, output, to an output device, an indication that the first object is not properly arranged for the biodecontamination process. The instructions, when executed, also cause one or more processors to, for a second area in the one or images that includes the second region of the load, apply a second set of rules to the second area in the one or more images to verify that the second object is properly arranged for the biodecontamination process, wherein the second set of rules is different than the first set of rules, and, in response to determining that the second object violates at least one rule in the second set of rules, output, to the output device, an indication that the second object is not properly arranged for the biodecontamination process.

In another example, the disclosure is directed to a method for performing any of the techniques of this disclosure.

In another example, the disclosure is directed to a device configured to perform any of the techniques of any of the techniques this disclosure.

In another example, the disclosure is directed to an apparatus comprising means for performing any of the techniques this disclosure.

In another example, the disclosure is directed to a non-transitory computer-readable storage medium having stored thereon instructions that, when executed, cause one or more processors of a computing device to perform any of the techniques this disclosure.

In another example, the disclosure is directed to a system comprising one or more computing devices configured to perform any of the techniques this disclosure.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

The following drawings are illustrative of particular examples of the present disclosure and therefore do not limit the scope of the invention. The drawings are not necessarily to scale, though examples can include the scale illustrated, and are intended for use in conjunction with the explanations in the following detailed description wherein like reference characters denote like elements. Examples of the present disclosure will hereinafter be described in conjunction with the appended drawings.

FIG. 1 is a block diagram illustrating a chamber with a camera system and a computing device configured to capture and analyze images to determine whether a chamber is properly arranged, in accordance with one or more techniques of this disclosure.

FIG. 2 is a block diagram illustrating a more detailed example of a computing device configured to perform the techniques described herein.

FIG. 3 is an example view of an image captured by a camera system of an interior of a chamber, in accordance with one or more techniques of this disclosure.

FIG. 4 is a sequence of images segmenting an image of an interior of a chamber into regions and the subsequent analysis of the images to determine whether the chamber is properly arranged, in accordance with one or more techniques of this disclosure.

FIG. 5 is a flow diagram illustrating an example process for capturing and analyzing images to determine whether a chamber is properly arranged, in accordance with one or more techniques of this disclosure, or indicate is properly arranged.

FIG. 6 is an example view of a validated load prepared on a cart outside of a biodecontamination chamber, in accordance with one or more techniques of this disclosure.

### DETAILED DESCRIPTION

The following detailed description is exemplary in nature and is not intended to limit the scope, applicability, or configuration of the techniques or systems described herein in any way. Rather, the following description provides some practical illustrations for implementing examples of the techniques or systems described herein. Those skilled in the art will recognize that many of the noted examples have a variety of suitable alternatives.

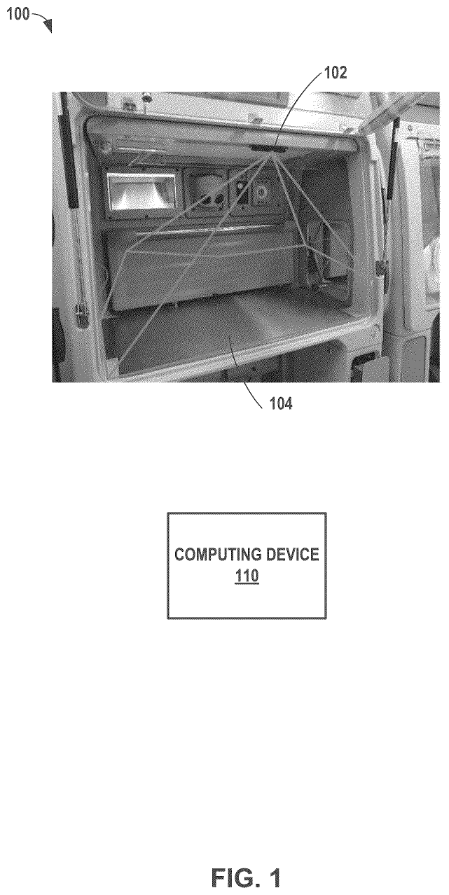

FIG. 1 is a block diagram illustrating system 100 with chamber 104 with sensor system 102 and computing device 110 configured to capture and analyze images to determine whether a chamber is properly arranged, in accordance with one or more techniques of this disclosure.

Sensor system 102 may be any one or more sensors that are capable of capturing one or more images of a load, such as one or more cameras, LiDAR sensors, laser sensors, radio frequency identification (RFID) proximity sensors, ultra-wideband (UWB) radar, or other computer vision systems capable of capturing one or more images, either as static images or as a sequence of images in a video stream or video file, and transmitting those one or more images to computing device 110. In some instances, sensor system 102 may also receive inputs from computing device 110 such that computing device 110 controls sensor system 102, or lighting within the chamber 104. Optical and/or non-optical spectrum devices may be used.

Biodecontamination is a process that inactivates microorganisms. It involves exposing the surface of objects within the chamber 104, the chamber itself and it's environment with a biodecontamination agent which may be in liquid, vapor, gaseous or light form using pre-validated parameters appropriate to the technology. Computing device 110 may be any computer with the processing power required to adequately execute the techniques described herein. For instance, computing device 110 may be any one or more of a mobile computing device (e.g., a smartphone, a tablet computer, a laptop computer, etc.), a desktop computer, a smarthome component (e.g., a computerized appliance, a home security system, a control panel for home components, a lighting system, a smart power outlet, etc.), an integrated computer system, a vehicle, a wearable computing device (e.g., a smart watch, computerized glasses, a heart monitor, a glucose monitor, smart headphones, etc.), a virtual reality/augmented reality/extended reality (VR/AR/XR) system, a video game or streaming system, a network modem, router, or server system, or any other computerized device that may be configured to perform the techniques described herein.

The biodecontamination process typically follows a specific sets of rules, potentially including: objects must not touch each other, be placed on flat surfaces such as the floor, obscure the biodecontaminants entry and circulation within the chamber, be of high or low temperature compared to the ambient temperature. If any of these rules are broken, the objects within chamber 104 may not be properly biodecontaminated, ultimately potentially resulting in product being contaminated. In some instances, these “rules” are indicated by previously validated load images, where the previously validated load images indicate positions of objects, spacing between objects, and any other “rule” that a load to be placed in a biodecontamination unit should be subjected to. Image comparison techniques may be utilized to compare the image of the load to be analyzed to the previously validated load images, and a load may be approved so long as the load is within a particular threshold of similarity as the previously validated load image.

In accordance with the techniques of this disclosure, computing device 110 may control sensor system 102 to capture one or more images of chamber 104 if a load is located in chamber 104, although other instances of the techniques described herein may include sensor system 102 being disconnected from system 100 and the load being analyzed prior to being loaded in chamber 104. The load may include at least a first object in a first region and a second object in a second region. The one or more captured images may each include at least the first region of the load and the second region of the load. For a first area in the one or more images that includes the first region of the load, computing device 110 may apply a first set of rules to the first area of the one or more images to verify that the first object is properly arranged for a biodecontamination process. In response to determining that the first object violates at least one rule in the first set of rules, computing device 110 may output, to an output device, an indication that the first object is not properly arranged for the biodecontamination process. For the purposes of this disclosure, chamber 104 being “properly arranged” means that objects that are expected to be in the load are present in the one or more images, that the objects are in the expected positions, both absolutely and relative to other objects in the load, and that no objects that are not supposed to be in the load are present in the load (e.g., when compared to a master image of a valid, properly arranged load).

For a second area in the one or images that includes the second region of the load, computing device 110 may apply a second set of rules to the second area of the one or more images to verify that the second object is properly arranged for the biodecontamination process, wherein the second set of rules is different than the first set of rules. In response to determining that the second object violates at least one rule in the second set of rules, computing device 110 may output, to the output device, an indication that the second object is not properly arranged for the biodecontamination process.

Rather than a manual comparison to a printed picture of the validated load positioning, the digital vision system described herein, using a sensor system, may provide a standardized load quality record. The automated verification of object arrangement within a load for biodecontamination processes, reducing human error and ensuring consistent compliance with predefined rules for object placement, thereby ensuring the efficacy of the biodecontamination process. Additionally, the techniques described herein may quickly and accurately assess load compliance, increasing consistency and decreasing the amount of time users spend loading and validating the load for a biodecontamination chamber. Furthermore, using the techniques described herein may remove the supervisor from the room, enabling them to focus on other activities and increasing the efficiency of the entire manufacturing process.

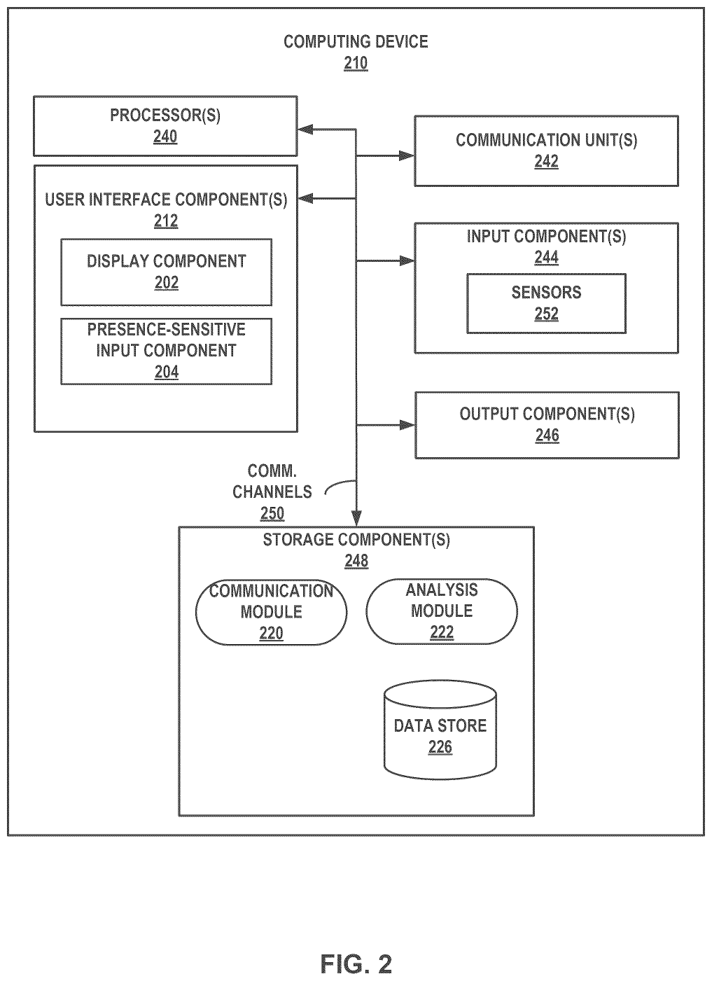

FIG. 2 is a block diagram illustrating a more detailed example of a computing device configured to perform the techniques described herein. Computing device 210 of FIG. 2 is described below as an example of computing device 110 of FIG. 1. FIG. 2 illustrates only one particular example of computing device 210, and many other examples of computing device 210 may be used in other instances and may include a subset of the components included in example computing device 210 or may include additional components not shown in FIG. 2.

Computing device 210 may be any computer with the processing power required to adequately execute the techniques described herein. For instance, computing device 210 may be any one or more of a mobile computing device (e.g., a smartphone, a tablet computer, a laptop computer, etc.), a desktop computer, a smarthome component (e.g., a computerized appliance, a home security system, a control panel for home components, a lighting system, a smart power outlet, etc.), an integrated computer system, a vehicle, a wearable computing device (e.g., a smart watch, computerized glasses, a heart monitor, a glucose monitor, smart headphones, etc.), a virtual reality/augmented reality/extended reality (VR/AR/XR) system, a video game or streaming system, a network modem, router, or server system, or any other computerized device that may be configured to perform the techniques described herein.

As shown in the example of FIG. 2, computing device 210 includes user interface components (UIC) 212, one or more processors 240, one or more communication units 242, one or more input components 244, one or more output components 246, and one or more storage components 248. UIC 212 includes display component 202 and presence-sensitive input component 204. Storage components 248 of computing device 210 include communication module 220, analysis module 222, and data store 226.

One or more processors 240 may implement functionality and/or execute instructions associated with computing device 210 to analyze images from a camera in the chamber. That is, processors 240 may implement functionality and/or execute instructions associated with computing device 210 to receive images from a camera and apply various sets of rules to different portions of the image to verify that a is properly arranged for a biodecontamination process.

Examples of processors 240 include any combination of application processors, display controllers, auxiliary processors, one or more sensor hubs, and any other hardware configured to function as a processor, a processing unit, or a processing device, including dedicated graphical processing units (GPUs). Modules 220 and 222 may be operable by processors 240 to perform various actions, operations, or functions of computing device 210. For example, processors 240 of computing device 210 may retrieve and execute instructions stored by storage components 248 that cause processors 240 to perform the operations described with respect to modules 220 and 222. The instructions, when executed by processors 240, may cause computing device 210 to receive images from a camera and apply various sets of rules to different portions of the image to verify that a load is properly arranged for a biodecontamination process.

Communication module 220 may execute locally (e.g., at processors 240) to provide functions associated with receiving images from a camera, outputting image previews, and outputting notifications of improperly arranged loads. In some examples, communication module 220 may act as an interface to a remote service accessible to computing device 210. For example, communication module 220 may be an interface or application programming interface (API) to a remote server that receives images from a camera, outputs image previews, and outputs notifications of improperly arranged loads.

In some examples, analysis module 222 may execute locally (e.g., at processors 240) to provide functions associated with analyzing the images received by communication module 220 according to rules 226 to determine if the load is properly arranged. In some examples, analysis module 222 may act as an interface to a remote service accessible to computing device 210. For example, analysis module 222 may be an interface or application programming interface (API) to a remote server that analyzes the images received by communication module 220 according to rules 226 to determine if the load is properly arranged.

One or more storage components 248 within computing device 210 may store information for processing during operation of computing device 210 (e.g., computing device 210 may store data accessed by modules 220 and 222 during execution at computing device 210). In some examples, storage component 248 is a temporary memory, meaning that a primary purpose of storage component 248 is not long-term storage. Storage components 248 on computing device 210 may be configured for short-term storage of information as volatile memory and therefore not retain stored contents if powered off. Examples of volatile memories include random access memories (RAM), dynamic random access memories (DRAM), static random access memories (SRAM), and other forms of volatile memories known in the art.

Storage components 248, in some examples, also include one or more computer-readable storage media. Storage components 248 in some examples include one or more non-transitory computer-readable storage mediums. Storage components 248 may be configured to store larger amounts of information than typically stored by volatile memory. Storage components 248 may further be configured for long-term storage of information as non-volatile memory space and retain information after power on/off cycles. Examples of non-volatile memories include magnetic hard discs, optical discs, floppy discs, flash memories, or forms of electrically programmable memories (EPROM) or electrically erasable and programmable (EEPROM) memories. Storage components 248 may store program instructions and/or information (e.g., data) associated with modules 220 and 222 and data store 226. Storage components 248 may include a memory configured to store data or other information associated with modules 220 and 222 and data store 226.

Communication channels 250 may interconnect each of the components 212, 240, 242, 244, 246, and 248 for inter-component communications (physically, communicatively, and/or operatively). In some examples, communication channels 250 may include a system bus, a network connection, an inter-process communication data structure, or any other method for communicating data.

One or more communication units 242 of computing device 210 may communicate with external devices via one or more wired and/or wireless networks by transmitting and/or receiving network signals on one or more networks. Examples of communication units 242 include a network interface card (e.g., such as an Ethernet card), an optical transceiver, a radio frequency transceiver, a GPS receiver, a radio-frequency identification (RFID) transceiver, a near-field communication (NFC) transceiver, or any other type of device that can send and/or receive information. Other examples of communication units 242 may include short wave radios, cellular data radios, wireless network radios, as well as universal serial bus (USB) controllers.

One or more input components 244 of computing device 210 may receive input. Examples of input are tactile, audio, and video input. Input components 244 of computing device 210, in one example, include a presence-sensitive input device (e.g., a touch sensitive screen, a PSD), mouse, keyboard, voice responsive system, camera, microphone or any other type of device for detecting input from a human or machine. In some examples, input components 244 may include one or more sensor components (e.g., sensors 252). Sensors 252 may include one or more biometric sensors (e.g., fingerprint sensors, retina scanners, vocal input sensors/microphones, facial recognition sensors, cameras), one or more location sensors (e.g., GPS components, Wi-Fi components, cellular components), one or more temperature sensors, one or more movement sensors (e.g., accelerometers, gyros), one or more pressure sensors (e.g., barometer), one or more ambient light sensors, and one or more other sensors (e.g., infrared proximity sensor, hygrometer sensor, and the like). Other sensors, to name a few other non-limiting examples, may include a radar sensor, a lidar sensor, a sonar sensor, a heart rate sensor, magnetometer, glucose sensor, olfactory sensor, compass sensor, or a step counter sensor.

One or more output components 246 of computing device 210 may generate output in a selected modality. Examples of modalities may include a tactile notification, audible notification, visual notification, machine generated voice notification, or other modalities. Output components 246 of computing device 210, in one example, include a presence-sensitive display, a sound card, a video graphics adapter card, a speaker, a cathode ray tube (CRT) monitor, a liquid crystal display (LCD), a light emitting diode (LED) display, an organic LED (OLED) display, a virtual/augmented/extended reality (VR/AR/XR) system, a three-dimensional display, or any other type of device for generating output to a human or machine in a selected modality.

UIC 212 of computing device 210 may include display component 202 and presence-sensitive input component 204. Display component 202 may be a screen, such as any of the displays or systems described with respect to output components 246, at which information (e.g., a visual indication) is displayed by UIC 212 while presence-sensitive input component 204 may detect an object at and/or near display component 202.

While illustrated as an internal component of computing device 210, UIC 212 may also represent an external component that shares a data path with computing device 210 for transmitting and/or receiving input and output. For instance, in one example, UIC 212 represents a built-in component of computing device 210 located within and physically connected to the external packaging of computing device 210 (e.g., a screen on a mobile phone). In another example, UIC 212 represents an external component of computing device 210 located outside and physically separated from the packaging or housing of computing device 210 (e.g., a monitor, a projector, etc. that shares a wired and/or wireless data path with computing device 210).

UIC 212 of computing device 210 may detect two-dimensional and/or three-dimensional gestures as input from a user of computing device 210. For instance, a sensor of UIC 212 may detect a user's movement (e.g., moving a hand, an arm, a pen, a stylus, a tactile object, etc.) within a threshold distance of the sensor of UIC 212. UIC 212 may determine a two or three-dimensional vector representation of the movement and correlate the vector representation to a gesture input (e.g., a hand-wave, a pinch, a clap, a pen stroke, etc.) that has multiple dimensions. In other words, UIC 212 can detect a multi-dimension gesture without requiring the user to gesture at or near a screen or surface at which UIC 212 outputs information for display. Instead, UIC 212 can detect a multi-dimensional gesture performed at or near a sensor which may or may not be located near the screen or surface at which UIC 212 outputs information for display.

In accordance with the techniques of this disclosure, communication module 220 may control one or more sensors to capture one or more images of a load, such as any combination of one or more cameras, one or more LiDAR sensors, or any other computer vision system. The imaging area may contain a load that includes at least a first object in a first region of the load and a second object in a second region of the load, wherein the one or more images each include at least the first region of the load and the second region of the load.

In some instances, prior to capturing the image, communication module 220 may output, for display on a display device, a semi-transparent overlay on top of an image preview of the camera, wherein the semi-transparent overlay depicts an approved load arrangement for each of the first region and the second region of the load. Enhancing user guidance during load preparation by displaying a semi-transparent overlay depicting approved load arrangements may reduce errors and improve compliance with validated configurations. In other instances, prior to capturing the image, communication module 220 may output, for display on a display device, a pre-determined image side-by-side along with an image preview of the camera, wherein the pre-determined image depicts an approved load arrangement for each of the first region and the second region of the load. Provisioning visual comparison aids by displaying a pre-determined image alongside the camera preview may assist users in achieving correct load arrangements and minimize deviations from validated configurations. The display device may be one or more of an external display device and an image preview screen of the camera. The flexibility in display options may allow the use of various display devices to present overlays or comparison images, thereby accommodating different user environments and equipment setups.

For a first area in the one or more images that includes the first region of the load, analysis module 222 may apply a first set of rules to the first area in the one or more images to verify that the first object is properly arranged in or for the chamber. In response to analysis module 222 determining that the first object violates at least one rule in the first set of rules, communication module 220 may output, to an output device, an indication that the first object is not properly arranged for the biodecontamination process. In some instances, the indication that the first region is not properly arranged for the chamber may be any one or more of a colored highlight of the first region in the image, an audible indication of the first region not being properly arranged, an explicit stoppage of a biodecontamination unit performing the biodecontamination process prior to a release of a chemical into a chamber of the biodecontamination unit, a vibrotactile feedback indication of the first region not being properly arranged, and a textual notification of the first region not being properly arranged. The identification and communication of specific objects violating arrangement rules may provide detailed feedback to users for corrective actions, thus improving load preparation accuracy.

For the purposes of this disclosure, a “set of rules” can be any analytical tool where areas of one or more images can be checked to verify that the load is properly loaded. For instance, a “set of rules” can include a particular segment of a master image of a valid, properly arranged load where the area of the one or more images is compared to the particular segment of the master image to determine whether the load is within a threshold of similarity with the properly arranged load of the master image. In other instances, a “set of rules” may be in a different form, such as pre-programmed limitations.

In some instances, prior to determining that the first object violates at least one rule in the first set of rules and prior to determining that the second object violates at least one rule in the second set of rules, communication module 220 may perform a blocking action to stop the biodecontamination process from continuing until the applications of the first set of rules and the second set of rules by analysis module 222 are complete. The blocking actions could include any one or more of locking a door to a biodecontamination unit performing the biodecontamination process, outputting a user notification of the analysis being in process (e.g., to the biodecontamination unit, to a user device, or to UIC 212), aborting the biodecontamination process, disabling a required human machine interface (HMI) button, and blocking an advancement in the biodecontamination process.

In some instances, in applying the first set of rules to the first area in the one or more images, analysis module 222 may compare the first area in the one or more images to a model to generate an image difference metric for the first area in the one or more images. The model may include any one or more of a master image of a valid load, a plurality of images of valid loads for different sets of objects, and a generated image based on a plurality of images of valid loads. The utilization of diverse model types for image comparison may enhance the robustness and adaptability of the verification process to different load configurations and object sets.

Analysis module 222 may then compare the image difference metric to an image difference threshold. In response to the image difference metric exceeding the image difference threshold, analysis module 222 may determine that the first region is not properly arranged for the biodecontamination process. The precise assessment of load arrangement through image comparison techniques may generate an image difference metric to objectively evaluate compliance with validated configurations, thus ensuring accurate biodecontamination. The image difference metric may be any one or more of a score specific to the first object, a score specific to the first region, and a score specific to the image. The differentiation of image difference metrics for various aspects of the load may allow for detailed analysis and targeted feedback on specific objects or regions, thereby improving the accuracy of arrangement verification.

In some such instances, the image difference metric may be a first image difference metric of a plurality of image difference metrics. In such instances, in applying the second set of rules to the second area in the one or more images, analysis module 222 may compare the second area in the one or more images to the model to generate a second image difference metric, the second image difference metric being for the second area in the one or more images. Analysis module 222 may then compare the second image difference metric to a second image difference threshold. In response to the second image difference metric exceeding the second image difference threshold, analysis module 222 may determine that the second region is not properly arranged for the biodecontamination process. The application of distinct rules and thresholds for different regions of the load may enable tailored verification processes that account for unique arrangement requirements, thus ensuring biodecontamination efficacy.

In other such instances, in applying the second set of rules to the second area in the one or more images, analysis module 222 may compare the second area in the one or more images to the model. Based at least in part on the comparing of the second area in the one or more images to the model, analysis module 222 may update the image difference metric. Analysis module 222 may then compare the updated image difference metric to the image difference threshold. In response to the updated image difference metric exceeding the image difference threshold, analysis module 222 may determine that the load is not properly arranged for the biodecontamination process. The dynamic updating of image difference metrics based on ongoing comparisons may allow for real-time adjustments and feedback to ensure proper load arrangement, thereby enhancing process reliability.

For a second area in the one or images that includes the second region of the load, analysis module 222 may apply a second set of rules to the second area in the one or more images to verify that the second object is properly arranged for the biodecontamination process, wherein the second set of rules is different than the first set of rules. In response to analysis module 222 determining that the second object violates at least one rule in the second set of rules, communication module 220 may output, to the output device, an indication that the second object is not properly arranged for the biodecontamination process.

In some instances, in response to analysis module 222 verifying that each of the first object and the second object are properly arranged for the chamber, communication module 220 may output, to the output device, an indication for the biodecontamination process to proceed to a next step. Facilitating of process automation by providing an indication to proceed to the next step in the biodecontamination process once the load is verified as properly arranged may streamline operations and reduce manual intervention.

Additionally or alternatively, in response to analysis module 222 may verifying that each of the first object and the second object are properly arranged for the biodecontamination process, communication module 220 may send an activation signal to an external device to perform a next step in the biodecontamination process. Integrating an external device activation into the automated verification process may enable seamless transition to subsequent biodecontamination steps and improve operational efficiency.

In some instances, analysis module 222 may identify the first object in the first area in the one or more images. In response to analysis module 222 determining that the first object violates at least one rule in the first set of rules, communication module 220 may include an identity of the first object in the indication that the first region for the load is not properly arranged.

In some instances, the load further includes at least a third object in a third region of the load and a fourth object in a fourth region of the load. In such instances, the one or more images further each include at least the third region of the load and the fourth region of the load. For a third area in the one or more images that includes the third region of the load, analysis module 222 may apply a third set of rules to the third area in the one or more images to verify that the third object is properly arranged for the biodecontamination process, wherein the third set of rules is different than the first set of rules and the second set of rules. In response to analysis module 222 determining that the third object violates at least one rule in the third set of rules, communication module 220 may output, to the output device, an indication that the third object is not properly arranged for the biodecontamination process. For a fourth area in the one or more images that includes the fourth region of the load, analysis module 222 may apply a fourth set of rules to the fourth area in the one or more images to verify that the fourth object is properly arranged for the biodecontamination process, wherein the fourth set of rules is different than the first set of rules, the second set of rules, and the third set of rules. In response to analysis module 222 determining that the fourth object violates at least one rule in the fourth set of rules, communication module 220 may output, to the output device, an indication that the fourth object is not properly arranged for the biodecontamination process. The extension of verification processes to additional objects and regions within the load may accommodate complex arrangements and ensure comprehensive compliance with biodecontamination requirements.

In some instances, in response to communication module 220 outputting either the indication that the first region is not properly arranged for the biodecontamination process or that the second region is not properly arranged for the biodecontamination process, communication module 220 may receive an indication that a user has adjusted the load. In response to communication module 220 receiving the indication that the user has adjusted the load, communication module 220 may control the one or more sensors to capture a second set of one or more images of the load. For the first area in the second set of one or more images that includes the first region of the load, analysis module 222 may apply the first set of rules to the first area in the second set of one or more images to verify that the first object is properly arranged for the biodecontamination unit. In response to analysis module 222 determining that the first object violates at least one rule in the first set of rules, communication module 220 may output, to the output device, the indication that the first object is not properly arranged for the biodecontamination unit. For the second area in the second set of one or more images that includes the second region of the chamber, analysis module 222 may apply the second set of rules to the second area in the second set of one or more images to verify that the second object is properly arranged for the biodecontamination process. In response to analysis module 222 determining that the second object violates at least one rule in the second set of rules, communication module 220 may output, to an output device, an indication that the second object is not properly arranged for the biodecontamination process.

In some instances, in response to communication module 220 outputting either the indication that the first object is not properly arranged for the biodecontamination process or that the second object is not properly arranged for the biodecontamination process, communication module 220 may receive an indication of a user overriding a procedural block preventing progression to the next process step, such as locking a chamber or a barrier. In response to this user input, communication module 220 may activate the chamber or barrier. The provision of user override capabilities for procedural blocks may enable flexibility in process management and accommodate exceptional circumstances while maintaining control over biodecontamination steps.

In some instances, analysis module 222 may measure one or more load statistics, or statistics descriptive of the operations of the chamber and the efficacy of the loader of the chamber. For example, the one or more load statistics may include any one or more of a number of cycles until the one or more processors detect an approved load, a time to complete a loading of a chamber, a location of the chamber, a time, a date, user information, a test result for the chamber, a list, a summary, a compilation of image difference metrics, and a number of times where a process was overridden. Communication module 220 may report these statistics out to users or managers for further evaluation. The collection and analysis of load statistics may provide valuable insights into process performance and user interactions, thereby supporting continuous improvement and optimization of biodecontamination operations.

In some instances, the first set of rules includes an order in which objects must be loaded into the first region. In response to the first object being properly arranged into the first region, communication module 220 may control the one or more sensors to capture a second set of one or more images of an updated load that includes at least the load and a third object in the first region of the load. Analysis module 222 may apply the first set of rules to the first area in the one or more images to verify that the third object is properly arranged for the biodecontamination process. In response to analysis module 222 determining that the third object violates at least one rule in the first set of rules, communication module 220 may output, to an output device, an indication that the third object is not properly arranged for the biodecontamination unit. The enforcement of object loading order within specific regions may ensure adherence to validated configurations and enhancing the reliability of the biodecontamination process.

For the purposes of this disclosure, any area of the load may have any number of objects. While the process is described above with respect to a single object in a single area, or iterating the checking of the objects with each singular addition of an object, an area of the load may include two, three, four, or more objects, and analysis module may apply the various sets of rules to any number of objects located in the particular area. For any description of the techniques that relate to actions performed in analyzing a single object, the same techniques can be utilized to analyze any number of objects in that area at a same or substantially similar time (e.g., within the same set of operations).

Computing device 210 may be a local computing device performing calculations as a device physically connected to (or integrated into) a biodecontamination unit, a local computing device in direct wireless communication with the biodecontamination unit, or may be a server device receiving the data from the local sensors over a cellular or internet connection. However, computing device 210 may benefit from being a local computing device (or an edge computing device), as computing device 210 being local would reduce data transfer costs, improve compliance with regional or user-specific privacy requirements (e.g., GDPR or IP security), improve compliance with local manufacturing firewalls, and improve validation and reliability requirements.

For instance, pharmaceutical industries must validate all manufacturing processes. This means that cloud and continuously-learning applications could present a risk, as version changes must be controlled. Also network connectivity could delay production, if, for example a data connection is needed for advancement to a next step, but the network disconnects. As a result, local versions of software and hardware are preferable in these unique instances, although cloud- and server-based may still perform the techniques described herein adequately.

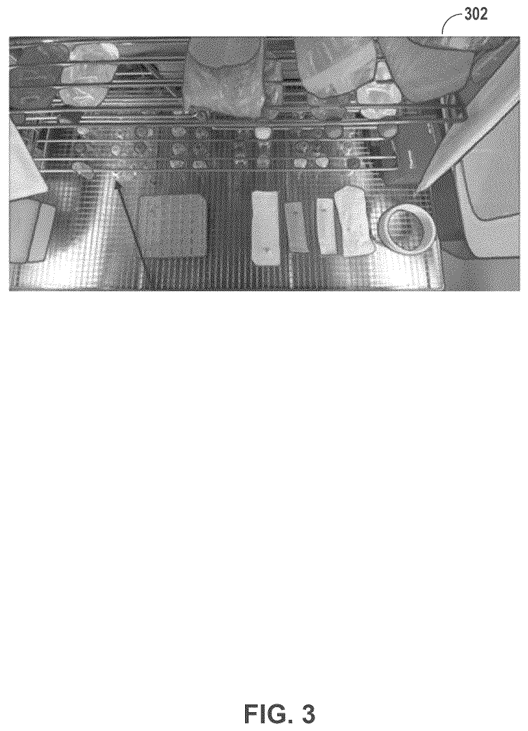

FIG. 3 is an example view of an image captured 302 by a camera system (e.g., sensor system 102) of an interior of a chamber (e.g., chamber 104), in accordance with one or more techniques of this disclosure. In image 302, each of a number of items are captured, identified, and graphically highlighted as part of the evaluation process of the load, such as chamber 104 of FIG. 1. In an outputted graphical user interface, each of the identified objects may be named by a computing device, and computing device 110 may further evaluate each object to verify that the respective object satisfies the applicable rules. If each object satisfies the applicable rules, then computing device 110 may output and indication of the objects satisfying the rules and an interactable element for a user to begin the biodecontamination process. If one or more of the highlighted objects fail to satisfy an applicable rule, computing device 110 may output a graphical indication of the particular object (or location of the particular object) and an indication that the particular object fails one of the applicable rules. In some instances, computing device 110 may also output the particular rule that was failed. It may also output an indication that the object is in the correct location.

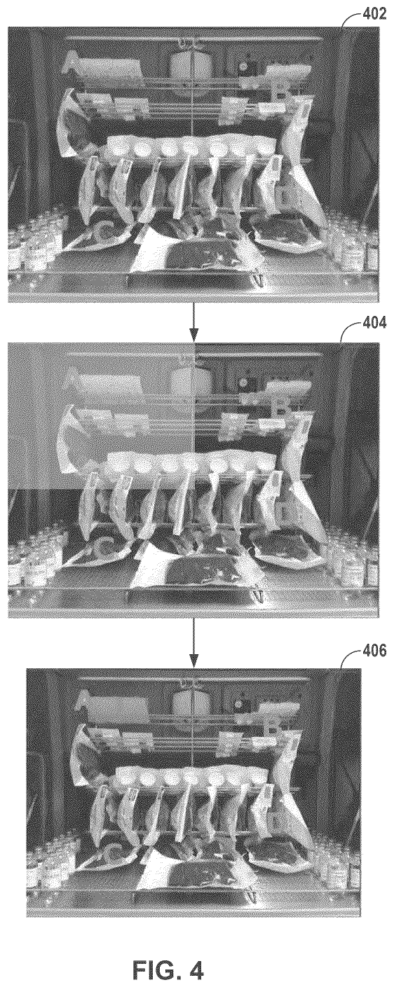

FIG. 4 is a sequence of images 402-406 segmenting an image of an interior of a chamber into regions and the subsequent analysis of the images to determine whether the load is properly arranged, in accordance with one or more techniques of this disclosure.

In image 402, a computing device breaks the image down into four regions, A, B, C, and D. For output, a computing device displays a semi-transparent, validated load over a video feed (e.g., a sequence of images captured live by a camera streaming to the computing device) to guide user load preparation. When prompted by the computing device, the camera may capture auditable load images, such as image 402.

If one of the regions has failed the validation check, the computing device may output any number of indications, including any one or more of an image difference score, a textual or audible alert (e.g., “something in the upper left quarter of the screen does not match”), or may highlight the failed region in a color, such as red or yellow.

In some instances, the computing device may even further output specific notifications for objects using optimized image recognition and interpretation. For example, the computing device may include object recognition to tell the user exactly what needs correction (e.g., “The highlighted object is wrong”), as shown in image 406. To utilize such a feature, the computing device may be trained with thousands of validation images, and may incorporate techniques for object segmentation or refined image difference scoring.

In the evaluation process, users may first perform manual quality control review while a computing device optimizes the algorithm with the latest data. This may include loading a validated image database with pre-approved validated loads in the form of images.

In instances where multiple load patterns are possible, a user or eSOP selects desired load pattern at the computing device. The computing device then displays the validated load pattern for the user, either as a semitransparent overlay or a side-by-side image on screen with a video of a load area.

The computing device may then perform a comparison between the actual load and the validated load from a database. This may be in real-time, or may be on demand after a user interaction, such as a selection of a “review load” button selected. Additionally, the comparison may be via entire image comparison, region-specific image similarity comparison, or machine learning object identification.

Based on the comparison, the computing device generates one or more scores (e.g., a full image score, stage scores, or partial scores) to rate the current load. The computing device may translate the score to an output for a user and/or a company. For example (from low to high recognition): -“Load not recognized”-“Load approved”-“Difference detected in upper left quadrant”. The computing device may output the textual translation and/or displayed the translated score as a colored area on image (e.g., “Object X incorrectly positioned” written and/or displayed as a colored object on the image).

The computational solution utilized to distinguish between correct and incorrect loading on the system may employ a Machine Learning (ML) model. The operation of the ML model, for instance, may follow the procedure outlined below:

Initially, an example of a “correct load” photograph may be provided to the system according to this provided example. The same “correct load” photograph is supplied to the ML model. After the system has completed loading the image, a picture of the load (referred to as “current load”) may be taken and supplied to the ML model.

The ML model may then compare the “correct load” and “current load” photographs using various Image Similarity metrics. Each of these metrics may focus on different aspects of the images and results in a numerical score.

Once all scores are calculated, a composite score may be created by averaging most of the scores into a new quantity termed “Mean Image Difference” (MID). Another score, namely the image “Mean Square Error” (MSE), may also be provided.

The MID score may be displayed as a percentage, with a range from 0 (completely dissimilar images) to 100 (identical images). The MSE score may be a natural number with possible values ranging from 0 (identical images) to arbitrarily high numbers (very different images). Based on the MID and MSE values, the ML model may provide the following outputs to the operator:

a) A MID score below a certain threshold (e.g., 80%) may result in a FAIL output by the ML model, meaning the two images differ significantly, indicating improper loading of system. The operator is instructed to correct the load and retry.

b) A MID score above a second threshold (e.g., 97.5%) may results in a PASS output by the ML model, The operator is given approval to proceed with sterilization.

c) A MID score between the two thresholds (e.g., between 80% and 97.5%) may be considered ambiguous. Within this range, an MSE score below 1 may result in a PASS output by the ML model, allowing the operator to proceed with sterilization. Conversely, if the MSE exceeds 1, the ML model may yield a “BORDERLINE” output. In this case, the operator is instructed to re-inspect the load, adjust it to more closely match the provided example load, and either retry the ML model inspection or proceed with sterilization.

Once the computing device outputs the results of the evaluation, any number of user interactions may be received. One such user interaction is that the user adjusts the load and repeats the process, with the pre-validated load displayed for user. Another interaction is that the user overrides the recognition output, signing and dating the instance of override.

Another such interaction is that the user ignores negative output. In this instance, a biodecontamination cycle may not start, a barrier opening may not be allowed, or other indicator preventing the user from progressing to the next step in the biodecontamination process. Another such interaction is that the user accepts “approved” output, potentially signing and dating the approved cycle. The load image captured may be used in training the model to further increase efficiency of the system. The computing device may then enable a biodecontamination cycle.

Throughout operation, a computing device may capture a number of statistics for the overall system. This could include any one or more of a number of cycles until the computing device detected an approved load, a time to complete an approved loading, BI/CI SN, a test location, and a test result.

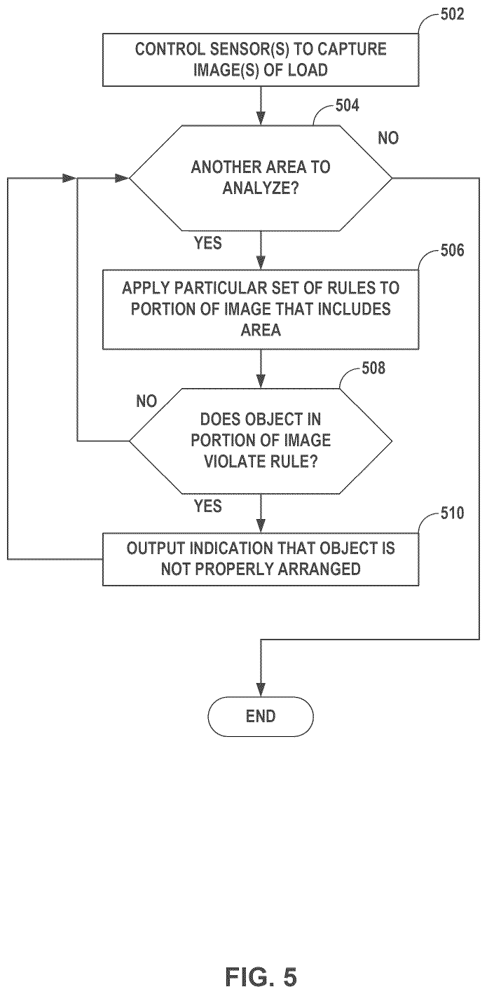

FIG. 5 is a flow chart illustrating an example mode of operation. The techniques of FIG. 5 may be performed by one or more processors of a computing device, such as system 100 of FIG. 1 and/or computing device 210 illustrated in FIG. 2. For purposes of illustration only, the techniques of FIG. 5 are described within the context of computing device 210 of FIG. 2, although computing devices having configurations different than that of computing device 210 may perform the techniques of FIG. 5.

In accordance with the techniques of this disclosure, communication module 220 may control one or more sensors to capture one or more images of a load, wherein the load contains at least a first object in a first region of the load and a second object in a second region of the load, wherein the one or more images each include at least the first region of the load and the second region of the load (502). For a first area in the one or more images that includes the first region of the load (YES branch of 504), analysis module 222 applies a first set of rules to the first portion of the image to verify that the first object is properly arranged in the load (506). Analysis module 222 determines whether the first set of rules are violated (508). If the portion of the image does not violate the first set of rules (NO branch of 508), analysis module 222 determines whether another area of the image is to be analyzed (504). In response to a portion of the image violating one of the set of rules (YES branch of 508), communication module 220 outputs, to an output device, an indication that the first object is not properly arranged for the biodecontamination process. If there is another portion of the image that is to be analyzed (YES branch of 504), analysis module 222 and communication module 220 repeat steps 506, 508, and 510 as necessary. If analysis module 222 has completed the analysis (NO branch of 504), then the instance of the process ends.

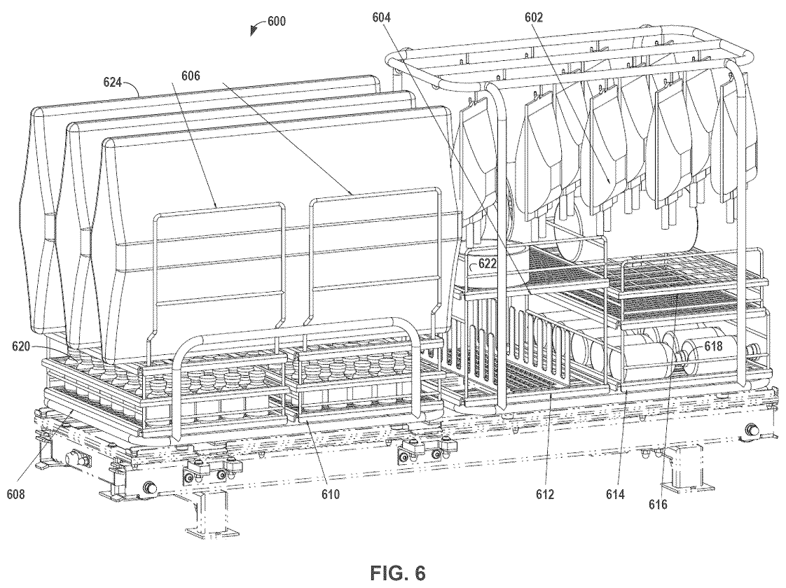

FIG. 6 is an example view of a validated load prepared on a cart 600 outside of a biodecontamination chamber, in accordance with one or more of the techniques described herein. In this particular validated load example, an assortment of transfusion bags 602, bottles 618, vials 620, cuffs 622, and other large objects 624 are arranged in dedicated, mesh rack (e.g., racks 606, 608, 610, 612, 614, and 616) to ensure maximum surface area exposure to bio-decontaminant, and to aid in reproducibility of the arrangement. This example cart is on a sliding track, enabling the user to prepare the validated load in the open air, then reproducibly transport the validated load into the appropriate chamber where the bio-decontamination process will occur.

Example 1. A method comprising: controlling, by one or more processors, one or more sensors to capture one or more images of a load, wherein the load contains at least a first object in a first region of the load and a second object in a second region of the load, wherein the one or more images each include at least the first region of the load and the second region of the load; for a first area in the one or more images that includes the first region of the load: applying, by the one or more processors, a first set of rules to the first area in the one or more images to verify that the first object is properly arranged for a biodecontamination process; and in response to determining that the first object violates at least one rule in the first set of rules, outputting, by the one or more processors and to an output device, an indication that the first object is not properly arranged for the biodecontamination process; and for a second area in the one or images that includes the second region of the load: applying, by the one or more processors, a second set of rules to the second area in the one or more images to verify that the second object is properly arranged for the biodecontamination process, wherein the second set of rules is different than the first set of rules; and in response to determining that the second object violates at least one rule in the second set of rules, outputting, by the one or more processors and to the output device, an indication that the second object is not properly arranged for the biodecontamination process.

Example 2. The method of Example 1, further comprising: in response to verifying that each of the first object and the second object in the load are properly arranged, outputting, by the one or more processors and to the output device, an indication for the biodecontamination process to proceed to a next step.

Example 3. The method of any one or more of Examples 1-2, further comprising: in response to verifying that each of the first object and the second object are properly arranged for the biodecontamination process, sending, by the one or more processors, an activation signal to an external device to perform a next step in the biodecontamination process.

Example 4. The method of any one or more of Examples 1-3, further comprising: prior to capturing the image, outputting, by the one or more processors and for display on a display device, a semi-transparent overlay on top of an image preview of the camera, wherein the semi-transparent overlay depicts an approved load arrangement for each of the first region and the second region of the load.

Example 5. The method of any one or more of Examples 1-4, further comprising: prior to capturing the image, outputting, by the one or more processors and for display on a display device, a pre-determined image side-by-side along with an image preview of the camera, wherein the pre-determined image depicts an approved load arrangement for each of the first region and the second region of the load.

Example 6. The method of any one or more of Examples 4-5, wherein the display device comprises one or more of an external display device and an image preview screen of the camera.

Example 7. The method of any one or more of Examples 1-6, wherein applying the first set of rules to the first area in the one or more images comprises: comparing, by the one or more processors, the first area in the one or more images to a model to generate an image difference metric for the first area in the one or more images; comparing, by the one or more processors, the image difference metric to an image difference threshold; and in response to the image difference metric exceeding the image difference threshold, determining, by the one or more processors, that the first region is not properly arranged for the biodecontamination process.

Example 8. The method of Example 7, wherein the model comprises one or more of: a master image of a valid load; a plurality of images of valid loads for different sets of objects; and a generated image based on a plurality of images of valid loads.

Example 9. The method of any one or more of Examples 7-8, wherein the image difference metric comprises one or more of: a score specific to the first object, a score specific to the first region, and a score specific to the image.

Example 10. The method of any one or more of Examples 7-9, wherein the image difference metric comprises a first image difference metric, and wherein applying the second set of rules to the second area in the one or more images comprises: comparing, by the one or more processors, the second area in the one or more images to the model to generate a second image difference metric, the second image difference metric being for the second area in the one or more images; comparing, by the one or more processors, the second image difference metric to a second image difference threshold; and in response to the second image difference metric exceeding the second image difference threshold, determining, by the one or more processors, that the second region is not properly arranged for the biodecontamination process.

Example 11. The method of any one or more of Examples 7-9, wherein applying the second set of rules to the second area in the one or more images comprises: comparing, by the one or more processors, the second area in the one or more images to the model; based at least in part on the comparing of the second area in the one or more images to the model, updating, by the one or more processors, the image difference metric; comparing, by the one or more processors, the updated image difference metric to the image difference threshold; and in response to the updated image difference metric exceeding the image difference threshold, determining, by the one or more processors, that the load is not properly arranged for the biodecontamination process.

Example 12. The method of any one or more of Examples 1-11, wherein the indication that the first region is not properly arranged for the biodecontamination process comprises one or more of: a colored highlight of the first region in the image, an audible indication of the first region not being properly arranged, an explicit stoppage of a biodecontamination unit performing the biodecontamination process prior to a release of a chemical into a chamber of the biodecontamination unit, a vibrotactile feedback indication of the first region not being properly arranged, and a textual notification of the first region not being properly arranged.

Example 13. The method of any one or more of Examples 1-12, wherein the load further comprises at least a third object in a third region of the load and a fourth object in a fourth region of the load, wherein the one or more images each further include at least the third region of the load and the fourth region of the load, and wherein the method further comprises: for a third area in the one or more images that includes the third region of the load: applying, by the one or more processors, a third set of rules to the third area in the one or more images to verify that the third object is properly arranged for the biodecontamination process, wherein the third set of rules is different than the first set of rules and the second set of rules; and in response to determining that the third object violates at least one rule in the third set of rules, outputting, by the one or more processors and to the output device, an indication that the third object is not properly arranged for the biodecontamination process; and for a fourth area in the one or more images that includes the fourth region of the load: applying, by the one or more processors, a fourth set of rules to the fourth area in the one or more images to verify that the fourth object is properly arranged for the biodecontamination process, wherein the fourth set of rules is different than the first set of rules, the second set of rules, and the third set of rules; and in response to determining that the fourth object violates at least one rule in the fourth set of rules, outputting, by the one or more processors and to the output device, an indication that the fourth object is not properly arranged for the biodecontamination process.

Example 14. The method of any one or more of Examples 1-13, further comprising: in response to outputting either the indication that the first region is not properly arranged for the biodecontamination process or that the second region is not properly arranged for the biodecontamination process, receiving, by the one or more processors, an indication that a user has adjusted the load; in response to receiving the indication that the user has adjusted the load: controlling, by the one or more processors, the one or more sensors to capture a second set of one or more images of the load; for the first area in the second set of one or more images that includes the first region of the load: applying, by the one or more processors, the first set of rules to the first area in the second set of one or more images to verify that the first object is properly arranged for the biodecontamination process; and in response to determining that the first object violates at least one rule in the first set of rules, outputting, by the one or more processors and to the output device, the indication that the first object is not properly arranged for the biodecontamination process; and for the second area in the second set of one or more images that includes the second region of the chamber: applying, by the one or more processors, the second set of rules to the second area in the second set of one or more images to verify that the second object is properly arranged for the biodecontamination process; and in response to determining that the second object violates at least one rule in the second set of rules, outputting, by the one or more processors and to an output device, an indication that the second object is not properly arranged for the biodecontamination process.

Example 15. The method of any one or more of Examples 1-14, further comprising: in response to outputting either the indication that the first object is not properly arranged for the biodecontamination process or that the second object is not properly arranged for the biodecontamination process, receiving, by the one or more processors, an indication of a user overriding a procedural block preventing progression to the next process step.

Example 16. The method of any one or more of Examples 1-15, further comprising: measuring, by the one or more processors, one or more load statistics, the one or more load statistics comprising one or more of: a number of cycles until the one or more processors detect an approved load, a time to complete a loading of a chamber, a location of the chamber, a time, a date, user information, a test result for the chamber, a list, a summary, a compilation of image difference metrics, and a number of times where a process was overridden.

Example 17. The method of any one or more of Examples 1-16, wherein the first set of rules includes an order in which objects must be loaded into the first region, and wherein the method further comprises, in response to the first object being properly arranged into the first region: controlling, by the one or more processors, the one or more sensors to capture a second set of one or more images of an updated load comprising at least the load and a third object in the first region of the load; applying, by the one or more processors, the first set of rules to the first area in the one or more images to verify that the third object is properly arranged for the biodecontamination process; and in response to determining that the third object violates at least one rule in the first set of rules, outputting, by the one or more processors and to an output device, an indication that the third object is not properly arranged for the biodecontamination process.

Example 18. The method of any one or more of Examples 1-17, further comprising: identifying, by the one or more processors, the first object in the first area in the one or more images; and in response to determining that the first object violates at least one rule in the first set of rules, including, by the one or more processors, an identity of the first object in the indication that the first region for the load is not properly arranged.

Example 19. The method of any one or more of Examples 1-18, further comprising, prior to determining that the first object violates at least one rule in the first set of rules and prior to determining that the second object violates at least one rule in the second set of rules: performing, by the one or more processors, a blocking action to stop the biodecontamination process from continuing until the applications of the first set of rules and the second set of rules are complete, wherein the blocking action comprises any one or more of: locking a door to a biodecontamination unit performing the biodecontamination process, outputting, to a user device, a user notification of the analysis being in process, aborting the biodecontamination process, disabling a required human machine interface (HMI) button, and blocking an advancement in the biodecontamination process.

Example 20. A system comprising: one or more sensors; and a computing device comprising one or more processors configured to: control the one or more sensors to capture one or more images of a load, wherein the load contains at least a first object in a first region of the load and a second object in a second region of the load, wherein the one or more images each include at least the first region of the load and the second region of the load; for a first area in the one or more images that includes the first region of the load: apply a first set of rules to the first area in the one or more images to verify that the first object is properly arranged for a biodecontamination process; and in response to determining that the first object violates at least one rule in the first set of rules, output, to an output device, an indication that the first object is not properly arranged for the biodecontamination process; and for a second area in the one or images that includes the second region of the load: apply a second set of rules to the second area in the one or more images to verify that the second object is properly arranged for the biodecontamination process, wherein the second set of rules is different than the first set of rules; and in response to determining that the second object violates at least one rule in the second set of rules, output, to the output device, an indication that the second object is not properly arranged for the biodecontamination process.

Example 21. The system of Example 20, wherein the one or more processors are further configured to: in response to verifying that each of the first object and the second object in the load are properly arranged, output, to the output device, an indication for the biodecontamination process to proceed to a next step.

Example 22. The system of any one or more of Examples 20-21, wherein the one or more processors are further configured to: in response to verifying that each of the first object and the second object are properly arranged for the biodecontamination process, send an activation signal to an external device to perform a next step in the biodecontamination process.

Example 23. The system of any one or more of Examples 20-22, wherein the one or more processors are further configured to: prior to capturing the image, output, for display on a display device, a semi-transparent overlay on top of an image preview of the camera, wherein the semi-transparent overlay depicts an approved load arrangement for each of the first region and the second region of the load.

Example 24. The system of any one or more of Examples 20-23, wherein the one or more processors are further configured to: prior to capturing the image, output, for display on a display device, a pre-determined image side-by-side along with an image preview of the camera, wherein the pre-determined image depicts an approved load arrangement for each of the first region and the second region of the load.

Example 25. The system of any one or more of Examples 23-24, wherein the display device comprises one or more of an external display device and an image preview screen of the camera.

Example 26. The system of any one or more of Examples 20-25, wherein the one or more processors being configured to apply the first set of rules to the first area in the one or more images comprises the one or more processors being configured to: compare the first area in the one or more images to a model to generate an image difference metric for the first area in the one or more images; compare the image difference metric to an image difference threshold; and in response to the image difference metric exceeding the image difference threshold, determine that the first region is not properly arranged for the biodecontamination process.

Example 27. The system of Example 26, wherein the model comprises one or more of: a master image of a valid load; a plurality of images of valid loads for different sets of objects; and a generated image based on a plurality of images of valid loads.

Example 28. The system of any one or more of Examples 26-27, wherein the image difference metric comprises one or more of: a score specific to the first object, a score specific to the first region, and a score specific to the image.

Example 29. The system of any one or more of Examples 26-28, wherein the image difference metric comprises a first image difference metric, and wherein the one or more processors being configured to apply the second set of rules to the second area in the one or more images comprises the one or more processors being configured to: compare the second area in the one or more images to the model to generate a second image difference metric, the second image difference metric being for the second area in the one or more images; compare the second image difference metric to a second image difference threshold; and in response to the second image difference metric exceeding the second image difference threshold, determine that the second region is not properly arranged for the biodecontamination process.

Example 30. The system of any one or more of Examples 26-28, wherein the one or more processors being configured to apply the second set of rules to the second area of the one or more images comprises the one or more processors being configured to: compare the second area in the one or more images to the model; based at least in part on the comparing of the second area in the one or more images to the model, update the image difference metric; compare the updated image difference metric to the image difference threshold; and in response to the updated image difference metric exceeding the image difference threshold, determine that the load is not properly arranged for the biodecontamination process.

Example 31. The system of any one or more of Examples 20-30, wherein the one or more processors are further configured to: identify the first object in the first area in the one or more images; and in response to determining that the first object violates at least one rule in the first set of rules, include an identity of the first object in the indication that the first region for the load is not properly arranged.

Example 32. The system of any one or more of Examples 20-31, wherein the indication that the first region is not properly arranged for the biodecontamination process comprises one or more of: a colored highlight of the first region in the image, an audible indication of the first region not being properly arranged, an explicit stoppage of a biodecontamination unit performing the biodecontamination process prior to a release of a chemical into a chamber of the biodecontamination unit, a vibrotactile feedback indication of the first region not being properly arranged, and a textual notification of the first region not being properly arranged.

Example 33. The system of any one or more of Examples 20-32, wherein the load further comprises at least a third object in a third region of the load and a fourth object in a fourth region of the load, wherein the one or more images each further include at least the third region of the load and the fourth region of the load, and wherein the one or more processors are further configured to: for a third area in the one or more images that includes the third region of the load: apply a third set of rules to the third area in the one or more images to verify that the third object is properly arranged for the biodecontamination process, wherein the third set of rules is different than the first set of rules and the second set of rules; and in response to determining that the third object violates at least one rule in the third set of rules, output, to the output device, an indication that the third object is not properly arranged for the biodecontamination process; and for a fourth area in the one or more images that includes the fourth region of the load: apply a fourth set of rules to the fourth area in the one or more images to verify that the fourth object is properly arranged for the biodecontamination process, wherein the fourth set of rules is different than the first set of rules, the second set of rules, and the third set of rules; and in response to determining that the fourth object violates at least one rule in the fourth set of rules, output, to the output device, an indication that the fourth object is not properly arranged for the biodecontamination process.

Example 34. The system of any one or more of Examples 20-33, wherein the one or more processors are further configured to: in response to outputting either the indication that the first region is not properly arranged for the biodecontamination process or that the second region is not properly arranged for the biodecontamination process, receive an indication that a user has adjusted the load; in response to receiving the indication that the user has adjusted the load: control the one or more sensors to capture a second set of one or more images of the load; for the first area in the second set of one or more images that includes the first region of the load: apply the first set of rules to the first area in the second set of one or more images to verify that the first object is properly arranged for the biodecontamination process; and in response to determining that the first object violates at least one rule in the first set of rules, output, to the output device, the indication that the first object is not properly arranged for the biodecontamination process; and for the second area in the second set of one or more images that includes the second region of the chamber: apply the second set of rules to the second area in the second set of one or more images to verify that the second object is properly arranged for the biodecontamination process; and in response to determining that the second object violates at least one rule in the second set of rules, output, to the output device, an indication that the second object is not properly arranged for the biodecontamination process.

Example 35. The system of any one or more of Examples 20-34, wherein the one or more processors are further configured to: in response to outputting either the indication that the first object is not properly arranged for the biodecontamination process or that the second object is not properly arranged for the biodecontamination process, receive an indication of a user overriding a procedural block preventing progression to the next process step.

Example 36. The system of any one or more of Examples 20-35, wherein the one or more processors are further configured to: measure one or more load statistics, the one or more load statistics comprising one or more of: a number of cycles until the one or more processors detect an approved load, a time to complete a loading of a chamber, a location of the chamber, a time, a date, user information, a test result for the chamber, a list, a summary, a compilation of image difference metrics, and a number of times where a process was overridden.

Example 37. The system of any one or more of Examples 20-36, wherein the first set of rules includes an order in which objects must be loaded into the first region, and wherein the one or more processors are further configured to, in response to the first object being properly arranged into the first region: control the one or more sensors to capture a second set of one or more images of an updated load comprising at least the load and a third object in the first region of the load; apply the first set of rules to the first area in the one or more images to verify that the third object is properly arranged for the biodecontamination process; and in response to determining that the third object violates at least one rule in the first set of rules, output, to an output device, an indication that the third object is not properly arranged for the biodecontamination process.

Example 38. The system of any one or more of Examples 20-37, wherein the one or more processors are further configured to, prior to determining that the first object violates at least one rule in the first set of rules and prior to determining that the second object violates at least one rule in the second set of rules: perform a blocking action to stop the biodecontamination process from continuing until the applications of the first set of rules and the second set of rules are complete, wherein the blocking action comprises any one or more of: lock a door to a biodecontamination unit performing the biodecontamination process, output a user notification of the analysis being in process, abort the biodecontamination process, disable a required human machine interface (HMI) button, and block an advancement in the biodecontamination process.

Example 39. A non-transitory computer-readable storage medium having stored thereon instructions that, when executed, cause one or more processors of a computing device to: control one or more sensors to capture one or more images of a load, wherein the load contains at least a first object in a first region of the load and a second object in a second region of the load, wherein the one or more images each include at least the first region of the load and the second region of the load; for a first area in the one or more images that includes the first region of the load: apply a first set of rules to the first area in the one or more images to verify that the first object is properly arranged for a biodecontamination process; and in response to determining that the first object violates at least one rule in the first set of rules, output, to an output device, an indication that the first object is not properly arranged for the biodecontamination process; and for a second area in the one or images that includes the second region of the load: apply a second set of rules to the second area in the one or more images to verify that the second object is properly arranged for the biodecontamination process, wherein the second set of rules is different than the first set of rules; and in response to determining that the second object violates at least one rule in the second set of rules, output, to the output device, an indication that the second object is not properly arranged for the biodecontamination process.

Example 40. The non-transitory computer-readable storage medium of Example 39, wherein the instructions further cause the one or more processors to: in response to verifying that each of the first object and the second object in the load are properly arranged, output, to the output device, an indication for the biodecontamination process to proceed to a next step.

Example 41. The non-transitory computer-readable storage medium of any one or more of Examples 39-40, wherein the instructions further cause the one or more processors to: in response to verifying that each of the first object and the second object are properly arranged for the biodecontamination process, send an activation signal to an external device to perform a next step in the biodecontamination process.

Example 42. The non-transitory computer-readable storage medium of any one or more of Examples 39-41, wherein the instructions further cause the one or more processors to: prior to capturing the image, output, for display on a display device, a semi-transparent overlay on top of an image preview of the camera, wherein the semi-transparent overlay depicts an approved load arrangement for each of the first region and the second region of the load.

Example 43. The non-transitory computer-readable storage medium of any one or more of Examples 39-42, wherein the instructions further cause the one or more processors to: prior to capturing the image, output, for display on a display device, a pre-determined image side-by-side along with an image preview of the camera, wherein the pre-determined image depicts an approved load arrangement for each of the first region and the second region of the load.

Example 44. The non-transitory computer-readable storage medium of any one or more of Examples 42-43, wherein the display device comprises one or more of an external display device and an image preview screen of the camera.

Example 45. The non-transitory computer-readable storage medium of any one or more of Examples 39-44, wherein the instructions that cause the one or more processors to apply the first set of rules to the first area in the one or more images comprise instructions that, when executed by the one or more processors, cause the one or more processors to: compare the first area in the one or more images to a model to generate an image difference metric for the first area in the one or more images; compare the image difference metric to an image difference threshold; and in response to the image difference metric exceeding the image difference threshold, determine that the first region is not properly arranged for the biodecontamination process.

Example 46. The non-transitory computer-readable storage medium of Example 45, wherein the model comprises one or more of: a master image of a valid load; a plurality of images of valid loads for different sets of objects; and a generated image based on a plurality of images of valid loads.

Example 47. The non-transitory computer-readable storage medium of any one or more of Examples 45-46, wherein the image difference metric comprises one or more of: a score specific to the first object, a score specific to the first region, and a score specific to the image.

Example 48. The non-transitory computer-readable storage medium of any one or more of Examples 45-47, wherein the image difference metric comprises a first image difference metric, and wherein the instructions that cause the one or more processors to apply the second set of rules to the second area in the one or more images comprise instructions that, when executed by the one or more processors, cause the one or more processors to: compare the second area in the one or more images to the model to generate a second image difference metric, the second image difference metric being for the second area in the one or more images; compare the second image difference metric to a second image difference threshold; and in response to the second image difference metric exceeding the second image difference threshold, determine that the second region is not properly arranged for the biodecontamination process.

Example 49. The non-transitory computer-readable storage medium of any one or more of Examples 45-47, wherein the instructions that cause the one or more processors to apply the second set of rules to the second area of the one or more images comprise instructions that, when executed by the one or more processors, cause the one or more processors to: compare the second area in the one or more images to the model; based at least in part on the comparing of the second area in the one or more images to the model, update the image difference metric; compare the updated image difference metric to the image difference threshold; and in response to the updated image difference metric exceeding the image difference threshold, determine that the load is not properly arranged for the biodecontamination process.

Example 50. The non-transitory computer-readable storage medium of any one or more of Examples 39-49, wherein the instructions further cause the one or more processors to: identify the first object in the first area in the one or more images; and in response to determining that the first object violates at least one rule in the first set of rules, include an identity of the first object in the indication that the first region for the load is not properly arranged.

Example 51. The non-transitory computer-readable storage medium of any one or more of Examples 39-50, wherein the indication that the first region is not properly arranged for the biodecontamination process comprises one or more of: a colored highlight of the first region in the image, an audible indication of the first region not being properly arranged, an explicit stoppage of a biodecontamination unit performing the biodecontamination process prior to a release of a chemical into a chamber of the biodecontamination unit, a vibrotactile feedback indication of the first region not being properly arranged, and a textual notification of the first region not being properly arranged.

Example 52. The non-transitory computer-readable storage medium of any one or more of Examples 39-51, wherein the load further comprises at least a third object in a third region of the load and a fourth object in a fourth region of the load, wherein the one or more images each further include at least the third region of the load and the fourth region of the load, and wherein the instructions further cause the one or more processors to: for a third area in the one or more images that includes the third region of the load: apply a third set of rules to the third area in the one or more images to verify that the third object is properly arranged for the biodecontamination process, wherein the third set of rules is different than the first set of rules and the second set of rules; and in response to determining that the third object violates at least one rule in the third set of rules, output, to the output device, an indication that the third object is not properly arranged for the biodecontamination process; and for a fourth area in the one or more images that includes the fourth region of the load: apply a fourth set of rules to the fourth area in the one or more images to verify that the fourth object is properly arranged for the biodecontamination process, wherein the fourth set of rules is different than the first set of rules, the second set of rules, and the third set of rules; and in response to determining that the fourth object violates at least one rule in the fourth set of rules, output, to the output device, an indication that the fourth object is not properly arranged for the biodecontamination process.

Example 53. The non-transitory computer-readable storage medium of any one or more of Examples 39-52, wherein the instructions further cause the one or more processors to: in response to outputting either the indication that the first region is not properly arranged for the biodecontamination process or that the second region is not properly arranged for the biodecontamination process, receive an indication that a user has adjusted the load; in response to receiving the indication that the user has adjusted the load: control the one or more sensors to capture a second set of one or more images of the load; for the first area in the second set of one or more images that includes the first region of the load: apply the first set of rules to the first area in the second set of one or more images to verify that the first object is properly arranged for the biodecontamination process; and in response to determining that the first object violates at least one rule in the first set of rules, output, to the output device, the indication that the first object is not properly arranged for the biodecontamination process; and for the second area in the second set of one or more images that includes the second region of the chamber: apply the second set of rules to the second area in the second set of one or more images to verify that the second object is properly arranged for the biodecontamination process; and in response to determining that the second object violates at least one rule in the second set of rules, output, to the output device, an indication that the second object is not properly arranged for the biodecontamination process.

Example 54. The non-transitory computer-readable storage medium of any one or more of Examples 39-53, wherein the instructions further cause the one or more processors to: in response to outputting either the indication that the first object is not properly arranged for the biodecontamination process or that the second object is not properly arranged for the biodecontamination process, receive an indication of a user overriding a procedural block preventing progression to the next process step.

Example 55. The non-transitory computer-readable storage medium of any one or more of Examples 39-54, wherein the instructions further cause the one or more processors to: measure one or more load statistics, the one or more load statistics comprising one or more of: a number of cycles until the one or more processors detect an approved load, a time to complete a loading of a chamber, a location of the chamber, a time, a date, user information, a test result for the chamber, a list, a summary, a compilation of image difference metrics, and a number of times where a process was overridden.

Example 56. The non-transitory computer-readable storage medium of any one or more of Examples 39-55, wherein the first set of rules includes an order in which objects must be loaded into the first region, and wherein the instructions further cause the one or more processors to, in response to the first object being properly arranged into the first region: control the one or more sensors to capture a second set of one or more images of an updated load comprising at least the load and a third object in the first region of the load; apply the first set of rules to the first area in the one or more images to verify that the third object is properly arranged for the biodecontamination process; and in response to determining that the third object violates at least one rule in the first set of rules, output, to an output device, an indication that the third object is not properly arranged for the biodecontamination process.

Example 57. The non-transitory computer-readable storage medium of any one or more of Examples 39-56, wherein the instructions further cause the one or more processors to, prior to determining that the first object violates at least one rule in the first set of rules and prior to determining that the second object violates at least one rule in the second set of rules: perform a blocking action to stop the biodecontamination process from continuing until the applications of the first set of rules and the second set of rules are complete, wherein the blocking action comprises any one or more of: lock a door to a biodecontamination unit performing the biodecontamination process, output a user notification of the analysis being in process, abort the biodecontamination process, disable a required human machine interface (HMI) button, and block an advancement in the biodecontamination process.

Example 58. A method for performing any of the techniques of any combination of Examples 1-57.

Example 59. A device configured to perform any of the techniques of any combination of Examples 1-57.

Example 60. An apparatus comprising means for performing any of the techniques of any combination of Examples 1-57.

Example 61. A non-transitory computer-readable storage medium having stored thereon instructions that, when executed, cause one or more processors of a computing device to perform the techniques of any combination of Examples 1-57.

Example 62. A system comprising one or more computing devices configured to perform the techniques of any combination of Examples 1-57.

Example 63. Any of the techniques described herein.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

It is contemplated that the various aspects, features, processes, and operations from the various embodiments may be used in any of the other embodiments unless expressly stated to the contrary. Certain operations illustrated may be implemented by a computer executing a computer program product on a non-transient, computer-readable storage medium, where the computer program product includes instructions causing the computer to execute one or more of the operations, or to issue commands to other devices to execute one or more operations.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, the functionality described herein may be provided within dedicated hardware and/or software modules configured for encoding and decoding, or incorporated in a combined codec. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a codec hardware unit or provided by a collection of interoperative hardware units, including one or more processors as described above, in conjunction with suitable software and/or firmware.

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as a pre-configured, stand-alone hardware element and/or as preprogrammed hardware elements (e.g., application specific integrated circuits, FPGAs, and digital signal processors), or other related components.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

While the various systems described above are separate implementations, any of the individual components, mechanisms, or devices, and related features and functionality, within the various system embodiments described in detail above can be incorporated into any of the other system embodiments herein.

The terms “about” and “substantially,” as used herein, refers to variation that can occur (including in numerical quantity or structure), for example, through typical measuring techniques and equipment, with respect to any quantifiable variable, including, but not limited to, mass, volume, time, distance, wavelength, frequency, voltage, current, and electromagnetic field. Further, there is certain inadvertent error and variation in the real world that is likely through differences in the manufacture, source, or precision of the components used to make the various components or carry out the methods and the like. The terms “about” and “substantially” also encompass these variations. The term “about” and “substantially” can include any variation of 5% or 10%, or any amount-including any integer-between 0% and 10%. Further, whether or not modified by the term “about” or “substantially,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range. Although the various embodiments have been described with reference to preferred implementations, persons skilled in the art will recognize that changes may be made in form and detail without departing from the spirit and scope thereof.

Various examples of the disclosure have been described. Any combination of the described systems, operations, or functions is contemplated. These and other examples are within the scope of the following claims.

1. A method comprising:
controlling, by one or more processors, one or more sensors to capture one or more images of a load, wherein the load contains at least a first object in a first region of the load and a second object in a second region of the load, wherein the one or more images each include at least the first region of the load and the second region of the load;
for a first area in the one or more images that includes the first region of the load:
applying, by the one or more processors, a first set of rules to the first area in the one or more images to verify that the first object is properly arranged for a biodecontamination process; and
in response to determining that the first object violates at least one rule in the first set of rules, outputting, by the one or more processors and to an output device, an indication that the first object is not properly arranged for the biodecontamination process; and
for a second area in the one or images that includes the second region of the load:
applying, by the one or more processors, a second set of rules to the second area in the one or more images to verify that the second object is properly arranged for the biodecontamination process, wherein the second set of rules is different than the first set of rules; and
in response to determining that the second object violates at least one rule in the second set of rules, outputting, by the one or more processors and to the output device, an indication that the second object is not properly arranged for the biodecontamination process.

2. The method of claim 1, further comprising:
in response to verifying that each of the first object and the second object in the load are properly arranged, outputting, by the one or more processors and to the output device, an indication for the biodecontamination process to proceed to a next step.

3. The method of claim 1, further comprising:
in response to verifying that each of the first object and the second object are properly arranged for the biodecontamination process, sending, by the one or more processors, an activation signal to an external device to perform a next step in the biodecontamination process.

4. The method of claim 1, further comprising:
prior to capturing the image, outputting, by the one or more processors and for display on a display device, a semi-transparent overlay on top of an image preview of the camera, wherein the semi-transparent overlay depicts an approved load arrangement for each of the first region and the second region of the load.

5. The method of claim 1, further comprising:
prior to capturing the image, outputting, by the one or more processors and for display on a display device, a pre-determined image side-by-side along with an image preview of the camera, wherein the pre-determined image depicts an approved load arrangement for each of the first region and the second region of the load.

6. The method of claim 5, wherein the display device comprises one or more of an external display device and an image preview screen of the camera.

7. The method of claim 1, wherein applying the first set of rules to the first area in the one or more images comprises:
comparing, by the one or more processors, the first area in the one or more images to a model to generate an image difference metric for the first area in the one or more images;
comparing, by the one or more processors, the image difference metric to an image difference threshold; and
in response to the image difference metric exceeding the image difference threshold, determining, by the one or more processors, that the first region is not properly arranged for the biodecontamination process.

8. The method of claim 7, wherein the model comprises one or more of:
a master image of a valid load;
a plurality of images of valid loads for different sets of objects; and
a generated image based on a plurality of images of valid loads.

9. The method of claim 7, wherein the image difference metric comprises one or more of:
a score specific to the first object,
a score specific to the first region, and
a score specific to the image.

10. The method of claim 7, wherein the image difference metric comprises a first image difference metric, and wherein applying the second set of rules to the second area in the one or more images comprises:
comparing, by the one or more processors, the second area in the one or more images to the model to generate a second image difference metric, the second image difference metric being for the second area in the one or more images;
comparing, by the one or more processors, the second image difference metric to a second image difference threshold; and
in response to the second image difference metric exceeding the second image difference threshold, determining, by the one or more processors, that the second region is not properly arranged for the biodecontamination process.

11. The method of claim 7, wherein applying the second set of rules to the second area in the one or more images comprises:
comparing, by the one or more processors, the second area in the one or more images to the model;
based at least in part on the comparing of the second area in the one or more images to the model, updating, by the one or more processors, the image difference metric;
comparing, by the one or more processors, the updated image difference metric to the image difference threshold; and
in response to the updated image difference metric exceeding the image difference threshold, determining, by the one or more processors, that the load is not properly arranged for the biodecontamination process.

12. The method of claim 1, wherein the indication that the first region is not properly arranged for the biodecontamination process comprises one or more of:
a colored highlight of the first region in the image,
an audible indication of the first region not being properly arranged,
an explicit stoppage of a biodecontamination unit performing the biodecontamination process prior to a release of a chemical into a chamber of the biodecontamination unit,
a vibrotactile feedback indication of the first region not being properly arranged, and
a textual notification of the first region not being properly arranged.

13. The method of claim 1, wherein the load further comprises at least a third object in a third region of the load and a fourth object in a fourth region of the load, wherein the one or more images each further include at least the third region of the load and the fourth region of the load, and wherein the method further comprises:
for a third area in the one or more images that includes the third region of the load:
applying, by the one or more processors, a third set of rules to the third area in the one or more images to verify that the third object is properly arranged for the biodecontamination process, wherein the third set of rules is different than the first set of rules and the second set of rules; and
in response to determining that the third object violates at least one rule in the third set of rules, outputting, by the one or more processors and to the output device, an indication that the third object is not properly arranged for the biodecontamination process; and
for a fourth area in the one or more images that includes the fourth region of the load:
applying, by the one or more processors, a fourth set of rules to the fourth area in the one or more images to verify that the fourth object is properly arranged for the biodecontamination process, wherein the fourth set of rules is different than the first set of rules, the second set of rules, and the third set of rules; and
in response to determining that the fourth object violates at least one rule in the fourth set of rules, outputting, by the one or more processors and to the output device, an indication that the fourth object is not properly arranged for the biodecontamination process.

14. The method of claim 1, further comprising:
in response to outputting either the indication that the first region is not properly arranged for the biodecontamination process or that the second region is not properly arranged for the biodecontamination process, receiving, by the one or more processors, an indication that a user has adjusted the load;
in response to receiving the indication that the user has adjusted the load:
controlling, by the one or more processors, the one or more sensors to capture a second set of one or more images of the load;
for the first area in the second set of one or more images that includes the first region of the load:
applying, by the one or more processors, the first set of rules to the first area in the second set of one or more images to verify that the first object is properly arranged for the biodecontamination process; and
in response to determining that the first object violates at least one rule in the first set of rules, outputting, by the one or more processors and to the output device, the indication that the first object is not properly arranged for the biodecontamination process; and
for the second area in the second set of one or more images that includes the second region of the chamber:
applying, by the one or more processors, the second set of rules to the second area in the second set of one or more images to verify that the second object is properly arranged for the biodecontamination process; and
in response to determining that the second object violates at least one rule in the second set of rules, outputting, by the one or more processors and to an output device, an indication that the second object is not properly arranged for the biodecontamination process.

15. The method of claim 1, further comprising:
in response to outputting either the indication that the first object is not properly arranged for the biodecontamination process or that the second object is not properly arranged for the biodecontamination process, receiving, by the one or more processors, an indication of a user overriding a procedural block preventing progression to the next process step.

16. The method of claim 1, further comprising:
measuring, by the one or more processors, one or more load statistics, the one or more load statistics comprising one or more of:
a number of cycles until the one or more processors detect an approved load,
a time to complete a loading of a chamber,
a location of the chamber,
a time,
a date,
user information,
a test result for the chamber,
a list,
a summary,
a compilation of image difference metrics, and
a number of times where a process was overridden.

17. The method of claim 1, wherein the first set of rules includes an order in which objects must be loaded into the first region, and wherein the method further comprises, in response to the first object being properly arranged into the first region:
controlling, by the one or more processors, the one or more sensors to capture a second set of one or more images of an updated load comprising at least the load and a third object in the first region of the load;
applying, by the one or more processors, the first set of rules to the first area in the one or more images to verify that the third object is properly arranged for the biodecontamination process; and
in response to determining that the third object violates at least one rule in the first set of rules, outputting, by the one or more processors and to an output device, an indication that the third object is not properly arranged for the biodecontamination process.

18. The method of claim 1, further comprising:
identifying, by the one or more processors, the first object in the first area in the one or more images; and
in response to determining that the first object violates at least one rule in the first set of rules, including, by the one or more processors, an identity of the first object in the indication that the first region for the load is not properly arranged.

19. The method of claim 1, further comprising, prior to determining that the first object violates at least one rule in the first set of rules and prior to determining that the second object violates at least one rule in the second set of rules:
performing, by the one or more processors, a blocking action to stop the biodecontamination process from continuing until the applications of the first set of rules and the second set of rules are complete,
wherein the blocking action comprises any one or more of:
locking a door to a biodecontamination unit performing the biodecontamination process,
outputting, to a user device, a user notification of the analysis being in process,
aborting the biodecontamination process,
disabling a required human machine interface (HMI) button, and
blocking an advancement in the biodecontamination process.

20. A system comprising:
one or more sensors; and
a computing device comprising one or more processors configured to:
control the one or more sensors to capture one or more images of a load, wherein the load contains at least a first object in a first region of the load and a second object in a second region of the load, wherein the one or more images each include at least the first region of the load and the second region of the load;
for a first area in the one or more images that includes the first region of the load:
apply a first set of rules to the first area in the one or more images to verify that the first object is properly arranged for a biodecontamination process; and
in response to determining that the first object violates at least one rule in the first set of rules, output, to an output device, an indication that the first object is not properly arranged for the biodecontamination process; and
for a second area in the one or images that includes the second region of the load:
apply a second set of rules to the second area in the one or more images to verify that the second object is properly arranged for the biodecontamination process, wherein the second set of rules is different than the first set of rules; and
in response to determining that the second object violates at least one rule in the second set of rules, output, to the output device, an indication that the second object is not properly arranged for the biodecontamination process.

21. A non-transitory computer-readable storage medium having stored thereon instructions that, when executed, cause one or more processors of a computing device to:
control one or more sensors to capture one or more images of a load, wherein the load contains at least a first object in a first region of the load and a second object in a second region of the load, wherein the one or more images each include at least the first region of the load and the second region of the load;
for a first area in the one or more images that includes the first region of the load:
apply a first set of rules to the first area in the one or more images to verify that the first object is properly arranged for a biodecontamination process; and
in response to determining that the first object violates at least one rule in the first set of rules, output, to an output device, an indication that the first object is not properly arranged for the biodecontamination process; and
for a second area in the one or images that includes the second region of the load:
apply a second set of rules to the second area in the one or more images to verify that the second object is properly arranged for the biodecontamination process, wherein the second set of rules is different than the first set of rules; and
in response to determining that the second object violates at least one rule in the second set of rules, output, to the output device, an indication that the second object is not properly arranged for the biodecontamination process.
